(19) United States
(12) Patent Application Publication
(10) Pub. No.: US 2007/0224675 A1
(43) Pub. Date: Sep. 27, 2007

(54) Expression Cassette

(76) Inventors: Kari Juhani Airenne, Kuopio (FI); Anssi Mahonen, Kuopio (FI); Seppo Yla-Herttuala, Kuopio (FI)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/578,452

(22) PCT Filed: May 20, 2005
(86) PCT No.: PCT/GB05/01984

§ 371 Date: Nov. 21, 2006

(30) Foreign Application Priority Data
May 20, 2004 (GB) 0411257.9

(51) Int. Cl. C12N 15/63

(52) U.S. Cl. 435/252.33; 435/320.1

(57) ABSTRACT

An expression cassette comprises targeting sites flanking a Cre fusion gene and a Cre coding sequence interrupted by an intron. This cassette can be used for the generation CreAoxP constructs and to reduce toxicity caused by the over-expression of Cre in target cells.

### FIELD OF THE INVENTION

This invention relates to an expression cassette.

### BACKGROUND OF THE INVENTION

Cre and other recombinases of the λ-integrase family have proven to be powerful tools for the manipulation of plant and vertebrate genomes. Each enzyme cleaves DNA at a specific target sequence and can ligate the newly exposed ends to the cleaved DNA at the second target sequence. Two components are required for the Cre-based recombination: 1) loxP, a 34 bp consensus sequence, and 2) Cre recombinase, the 38 kDa product of the bacteriophage P1 Cre gene. The nature of the recombination event caused by Cre depends on the relative orientation of the two loxP sites. DNA flanked by the loxP sites oriented in the same direction is circulated during the recombination, whereas DNA flanked by loxP sites that are oriented in opposite directions, is inverted. The Cre-lox system is described in U.S. Pat. No. 4,959,317.

Over-expression of Cre recombinase has been found to be toxic for mammalian cells. It has been reported that Cre is toxic for at least some human cell lines (kidney cell line 293 and osteosarcoma cell line U2OS) and for Drosophila cells, and causes phenotypic aberrations in plants. A reasonable explanation for all of these observations is that at least human, mouse, yeast and E. coli genomes contain a number of endogenous sequences that can be targets for Cre.

The toxicity of Cre depends upon its strand-cleavage activity. This was demonstrated by Silver et al, Mol. Cell. 8, 233-243 (2001), in which it is reported that Cre mutants, defective in DNA-cleavage activity, were not toxic compared to wild-type Cre; a method in which Cre excises the gene directing its own synthesis, once a critical level of expression required for the excision is reached, was effective in avoiding toxicity. More particularly, it was observed by Silver et al that, when 293xLac cells (a derivative of the human embryonic kidney cell line 293) were infected with a retrovirus encoding a Cre recombinase-GFP fusion protein, the virus caused cellular toxicity, whereas the virus expressing GFP alone did not caused such changes. Silver et al generated a self-excising system functional only in retroviral vectors. This system contains one lox 511 site at the 3′ LTR U3 region of the virus genome. This loxP site will be duplicated during virus production and flanks the Cre/GFP fusion gene, permitting the development of a negative feed-back of Cre expression.

Genes under promoters considered to be active only in eukaryotic cells may direct gene expression also in E. coli. Chloramphenicol acetyl transferase (CAT) with the human cytomegalovirus immediately-early gene region 1 promoter-enhancer (HCMV-IE) was demonstrated to be expressed in HB101 E. coli strain, and genes under the avian tumor virus promoter were shown to be expressed in bacteria; see Sauer, Nucleic Acids Res 24, 4608-4613 (1996), and Mitsialis et al, Gene 16, 217-225 (1981).

If used in bacteria, leaky expression can cause significant problems not only with toxic products but also for the cloning of Cre/loxP constructs. Since bacteria cannot splice introns, one strategy to stop leaky expression in E. coli is the insertion of an intron into the coding region of Cre gene; see Zuo et al, Nat. Biotechnol. 19, 157-161 (2001), Kaczmarczyk & Green, Nucleic Acids Res 29, E56 (2001), and Bunting et al, Genes Dev. 13, 1524-1528 (1999).

### SUMMARY OF THE INVENTION

The present invention is based in part on the observation that, when Cre under the chicken β-actin promoter (CAG) was expressed in E. coli, there were significant problems for the cloning of Cre/loxP constructs. To avoid these problems, this invention provides an all-in-one Cre expression system referred to herein as Silent Self-inactivating Cre (SSi-Cre). This may also be applicable to other recombinases of the λ-integrase family flanked by targeting sites.

In the particular system described herein and which illustrates the invention, non-toxic Cre expression is restricted to eukaryotic cells. The use of mutated loxP sequences makes the SSi-Cre system also fully compatible with double loxP targeting strategies. The system may contain a reporter system, to visualize Cre activity in mammalian cells by fluorescent microscopy. The SSi-Cre system thus offers a useful solution for the major technical problems associated with the use of Cre/loxP system.

### BRIEF DESCRIPTION OF THE DRAWINGS

FIG. 1 shows construction of the pSSi-Cre expression cassette. Cre-coding sequence was interrupted by the intron. The cre/int fusion gene was subcloned into pDsRed2-N1 to form pCreInt. The cre/int/DsRed fusion gene was cloned between two mutant loxP sites to form pSSi-Cre.

FIG. 2 shows that leaky Cre expression is possible in E. coli due to the Shine-Dalgamo-like sequence upstream of the Cre gene. A) The level of leaky Cre production is indicated in the agarose gel picture by the presence of a 2,300 bp band. B) Comparison of the sequence of pCre with Shine-Dalgamo sequence of E. coli. The ribosome is able to recognize the Shine-Dalgamo like sequence of the pCre before initiation codon (ATG). C) Deletion of Xho I site in pCre reduced the leaky transcription.

FIG. 3 shows that pSSi-Cre strategy allows non-toxic Cre-expression in transfected cells. Fluorescence microscope analysis of transfected 293 T cells. A-B; red fluorescent protein expression shows no toxicity. C-D; Over-expression of Cre/Dsred fusion protein is toxic for the cells. E-G; Controlled expression solves toxicity associated to over-expression of Cre/DsRed. H-J; Red fluorescence disappears by time due to the self-inactivation of Cre/Dsred. K-M; Mock transfected cells. Original magnification was ×200.

FIG. 4. pSSi-Cre activates gene expression in CHO cells. A) The silenced VEGF expression is activated by Cre-mediated excision of STOP cassette. B) Expressed VEGF was detected by human VEGF ELISA assay.

### DESCRIPTION OF PREFERRED EMBODIMENTS

As indicated in more detail below, experiments have showed that persistent high-level Cre-expression causes cellular toxicity in 293T cells could be eliminated by regulating the duration and intensity of Cre recombinase expression. It was also noticed that expression of the Cre gene in E. coli under the mammalian CAG promoter caused significant problems for the cloning of Cre/loxP constructs. These problems were solved by constructing a SSi-Cre cassette which is universally compatible with Cre/loxP-experiments.

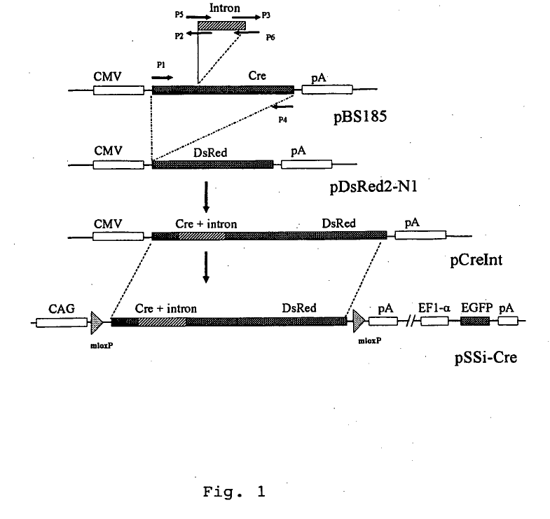

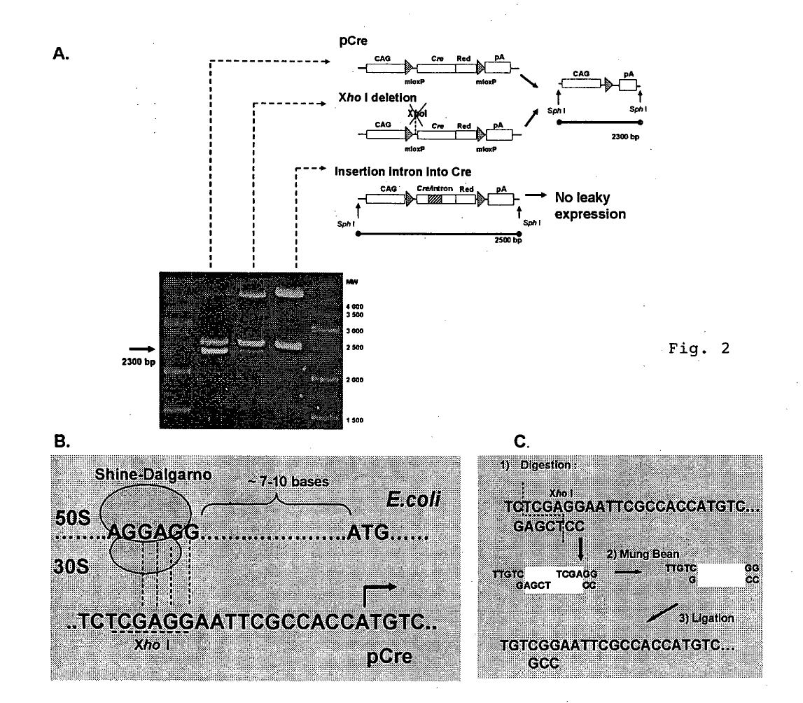

During the cloning procedure, it was not possible to construct a plasmid which contains both the Cre recombinase under a mammalian promoter and the DNA area flanked with the loxP recombination sites. In agarose gel electrophoresis, a strong 2,300 bp band was always detected, demonstrating the break-down of the construct (FIG. 2a). Without wishing to be bound by theory, this may be due to the background expression of Cre recombinase in E. coli. It is surprising that a promoter such as chicken CAG directs gene expression in E. coli. Since there are fundamental differences in the translation initiation between prokaryotic and eukaryotic cells, Cre translation should have not taken place to promote protein synthesis. Closer comparison of the sequence of pCre with Shine-Dalgamo sequence (see Kozak, Gene 234, 187-208 (1999)), which directs translation initiation in E. coli, showed that pCre contained a Shine-Dalgamo-like area just before the initiation codon of the Cre recombinase which might explain the leaky expression of Cre recombinase (FIG. 2b). To test this hypothesis, XhoI restriction site was deleted from the pCre (FIG. 2c). This deletion caused a significant reduction in Cre translation (FIG. 2c). Although the level of leaky expression was significantly reduced, a portion of the plasmids was still destroyed, creating a mixed population of intermediate constructs. To solve this problem, the Cre coding sequence was interrupted by a short mouse protamine intron to prevent bacterial expression of Cre (FIG. 1). This modification led to no leaky expression of Cre in E. coli, as shown in FIG. 2a. These findings clearly support the not so well-recognized ability of universal mammalian promoters to direct gene expression in bacteria.

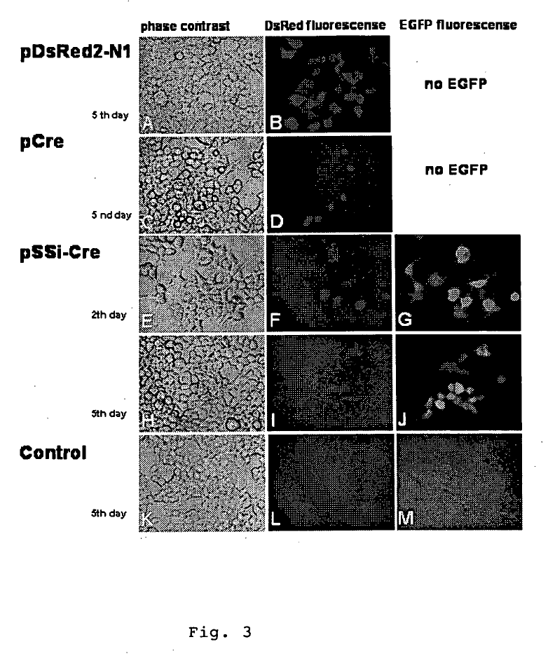

Cre recombinase expression resulted in cellular toxicity. 293T cells expressing Cre-DsRed fusion protein were rounded, unhealthy-looking and started to detach from the bottom of the wells as early as in 48 hour after transfection (FIG. 3). No such changes were observed in cells transfected with the red fluorescent protein encoding plasmid (pDsRed2-N1) or in mock treated cells. Thus, it is likely that the toxic effect seen in cells was Cre-dependent. The SSi-Cre system self-inactivates Cre expression as soon as possible after Cre production, to minimize intensity and duration of the Cre expression. Cre recombinase excises the fusion gene once the critical level of expression required for the excision has been reached. Unlike the idea of self-inactivating of Cre expression described by Silver et al, SSi-Cre contains both loxP sites by definition and is therefore compatible with all vectors.

To test the toxicity of the SSi-Cre system, 293T cells were transfected with the pSSi-Cre. 48 h after transfection, expression of the cre/int/DsRed fusion gene was observed as a faint red color in the transfected cells (FIG. 3F). However, as a result of the self-inactivation, the expression disappeared gradually during culturing. Five days after the transfection, the red colour was barely detectable (FIG. 3I), indicating that the cre/int/Dsred fusion gene was excised. To detect the transfected cells, pSSi-Cre construct contained the non-excisable EGFP expression unit beside the SSi-Cre cassette (FIG. 1). Cells transfected with the pSSi-Cre expressed green color and were looking healthy without any sign of toxicity (FIGS. 3G and 3J). This demonstrated that the strategy for Cre expression from the SSi-Cre cassette is feasible and non-toxic.

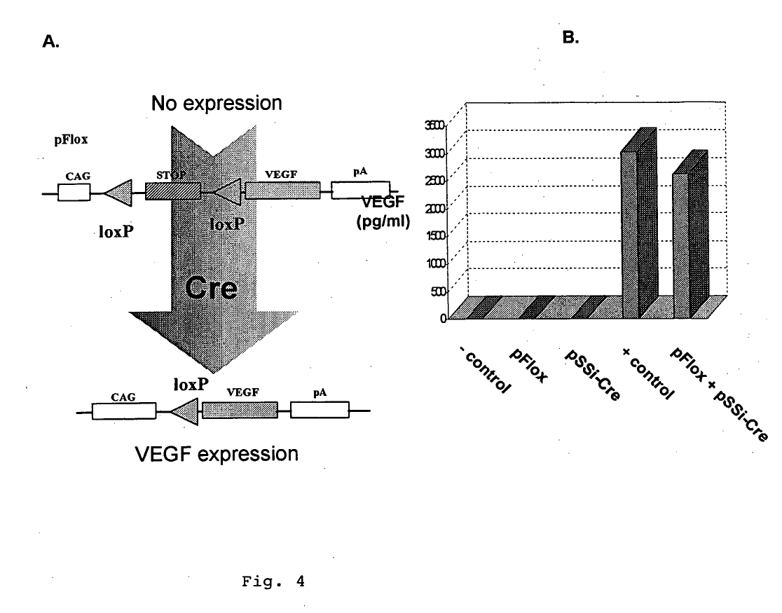

In order to investigate the functionality and compatibility of the pSSi-Cre with double-loxP experiments, pSSi-Cre was co-transfected into CHO cells with the pFlox plasmid which contains a wild type loxP-excisable STOP cassette. Excision of the STOP cassette activates VEGF expression which could be detected by ELISA assay (FIG. 4a). The level of Cre expression was sufficient to catalyze efficiently the recombination of DNA (FIG. 4b). This SSi-Cre cassette has been used successfully together with the wild-type loxP sites in the same plasmid without seeing any interference. These results prove that the intron-containing Cre/DsRed recombinase is functional and compatible with double-lox approaches.

The novel expression cassette thus enables a non-toxic expression of Cre in target cells. The SSi-Cre cassette restricts Cre expression only to eukaryotic cells, which allows strategies in which both the Cre recombinase gene and the loxP recombination sites are cloned in a single vector in E. coli. Since self-inactivation is mediated by modified loxP sites, multiple lox targeting experiments can be accomplished. SSi-Cre offers thus for the first time a solution to the major practical problems associated with Cre/loxP system in a convenient, single expression cassette which can be used in any desired context of that system.

The following Examples illustrate the invention.

Cloning of the Expression Cassette

Cre coding sequence was interrupted by a mouse protamine intron. This cre/int fusion gene was generated by a series of PCR's (FIG. 1). A 5 portion (nucleotides 1-432) from pBS185 plasmid (Life Technologies) was amplified using oligo P1 (5′-GTTACGAATTCGCCACCATGTCCAATTTACT GACCGT-3′) and oligo P2 (5′-CAGCCCTCTACTTACCTGGTCGAAATCAGTGCGTT-3′). A 3′ portion (nucleotides 433-1029) of Cre was amplified using oligo P3 (5′-TTCTTACCTTTCTAGGTTCGTTCACTCATGGAAAA-3′) and oligo P4 (5′-TAAGCAGATCTCCATCGCCATCTTCCAGCAGGC-3′). Mouse protamine intron (pAIV-11 as the template) was amplified using oligo P5 (5′-AC TGATTTCGACCAGGTAAGTAGAGGGCTGGGCTG-3′) and oligo P6 (5′-CATG AGTGAACGAACCTAGAAAGGTAAGAAAAGTG-3′). The cre/int fusion gene was made by using the three amplified PCR fragments as a template for a PCR with oligo P1 and oligo P4. This cre/int fusion gene was subcloned into EcoRI/BamHI sites of pDsRed2-N1 (BD Biosciences Clontech) to form pCreInt. In pCre plasmid, the intron was omitted (FIG. 1).

Modified two loxP sites (see Siegel et al, FEBSD Lett. 505, 467-473 (2001)) were cloned into the EcoRI site of pCAGGS and, between these sites, a cre/int/DsRed fusion gene. This SSi-Cre cassette (FIG. 1) together with EGFP expression unit was cloned into AvrII site in the pEvo, to form pSSi-Cre. The resulting plasmid was verified by DNA sequencing.

Deletion of Xho I Sites

pSSi-Cre was digested by XhoI, and single-strand extensions were removed using Mung Bean Nuclease (New England BioLabs, Inc., USA) according to the instructions of the manufacturer. Generated blunt ends were ligated using T4 DNA ligase (New England BioLabs, Inc., USA) by standard protocol.

Cell Culture and DNA Transfection

The pSSi-Cre plasmid was characterized in cell culture. Adherent 293 T cells were plated at a density of 200,000 cells per well. Plasmid/liposome transfections were done according to the instructions of the manufacturer (Fugene™, Roche, Basel, Switzerland). Transfected cells were examined by fluorescent microscopy.

ELISA Analysis

Functionality of the Cre recombinase was tested in CHO cells by co-transfection of pSSi-Cre plasmid with pFlox which contains a loxP-inactivated expression cassette for VEGF. The plasmid containing non-silenced VEGF gene under CMV promoter was used as a positive control. CHO cells were plated at 200,000 cells per well and FuGENE 6. Plasmid/liposome transfections were done according to the instructions of the manufacturer (Fugene™, Roche, Basel, Switzerland). Samples for human VEGF ELISA analysis (R&D Systems, Minneapolis, USA) were collected after 48 hours of culturing.

Results are shown in the drawings, and reported above.

1. An expression cassette comprising targeting sites flanking a Cre fusion gene and a Cre coding sequence interrupted by an intron.

2. The cassette according to claim 1, which additionally comprises a reporter gene, whereby Cre activity can be visualized.

3. The cassette according to claim 1, wherein the targeting sites comprise loxP.

4. The cassette according to claim 1, wherein a portion which directs translation initiation in E. coli is inactivated.

5. The cassette according to claim 1, wherein a portion having functional and/or structural homology to a Shine-Delgano sequence is inactivated.

6. The cassette according to claim 1, which comprises a mammalian promoter.

7. A method for generating Cre/loxP constructs and reducing toxicity caused by the over-expression of Cre in target cells wherein said method comprises the use of an expression cassette comprising targeting sites flanking a Cre fusion gene and a Cre coding sequence interrupted by an intron.

8. A host transformed with an expression cassette comprising targeting sites flanking a Cre fusion gene and a Cre coding sequence interrupted by an intron.

9. The host according to claim 8, which is E. coli.
